(19) United States
(12) Patent Application Publication
(10) Pub. No.: US 2008/0071145 A1
(43) Pub. Date: Mar. 20, 2008

(54) Support Clamp For Retractor Bar Stock Of Generally Rectangular Cross-Section

(75) Inventors: Todd M. BJORK, River Falls, WI (US); Christopher Lee BERG, Anoka, MN (US); Steven Karl Jacobson, St. Paul, MN (US)

Correspondence Address: SHEWCHUK IP SERVICES, 3356 SHERMAN CT. STE. 102, EAGAN, MN, 55121, US

(73) Assignee: LEVAHN INTELLECTUAL PROPERTY HOLDING COMPANY, LLC, St. Paul, MN (US)

(21) Appl. No.: 11/685,049

(22) Filed: Mar. 12, 2007

Related U.S. Application Data
(60) Provisional application No. 60/845,580, filed on Sep. 19, 2006.
(60) Provisional application No. 60/850,575, filed on Oct. 10, 2006.

(51) Int. Cl. A61B 1/32

(52) U.S. Cl. 600/227

(57) ABSTRACT

A clamp is used to attach generally rectangular bar stock, such as existing Bookwalter/Codman rings, to a post in a surgical retractor system. The clamp interacts with corners of the ring at any of a wide range of widths and thicknesses, while still retaining a simple and intuitive tightening action such as through a rotational collar which absorbs some of the clamping force as a hoop stress. In the preferred arrangement, the clamp makes a tripod top/planar bottom contact with the generally rectangular bar stock of the ring. An articulating joint permits best fold up of the clamp, with a ball shaft which is constrained within the bisecting plane of the joint clamp. A spherical nut is used on the clamp bolt in one of the clamps, which permits some change in orientation of the clamp bolt when the clamp bolt is tightened using a cammed handle.

### CROSS-REFERENCE TO RELATED APPLICATION(S)

This application claims priority from provisional application no. 60/845,580, filed Sep. 19, 2006, and also claims priority from provisional application no. 60/850,575, filed Oct. 10, 2006.

### BACKGROUND OF THE INVENTION

The present invention relates to the field of surgical tools, and particularly to the design and manufacture of surgical retractor systems. Surgical retractor systems are used during surgery to bias and hold tissue in a desired position. As one example, some surgical procedures require anterior access to the spine, through the patient's abdomen. Tissue such as skin, muscle, fatty tissue and interior organs needs to be held retracted to the side so the surgeon can obtain better access to the vertebrae structures of primary interest.

Such retractor assemblies may, for instance, include a frame element, commonly referred to as a “ring”, which is rigidly supported from the patient's bed above and around the surgical incision location, with a number of clamps and retractor blades to hold back tissue proximate to the surgical incision. One type of such retractor assembly is shown in the various patents of John R. Bookwalter et al., such as U.S. Pat. Nos. 4,254,763, 4,421,108, 4,424,724, 4,467,791, 5,375,481, 5,520,608, 6,241,659, 6,530,882 and 6,808,493, all incorporated by reference, as originally made and marketed by Codman & Shurtleff, Inc. of Randolph, Mass. Additional examples include those shown in U.S. Pat. Nos. 1,919,120, 1,963,173, 4,434,791, and 5,520,610, all incorporated by reference. In the Bookwalter/Codman system and in these other examples, the frame element is a flat ring of generally rectangular cross-section. The ring is held by a support post that clamps to the side rail of the operating table, so the ring is suspended in a plane above the surgical site.

The term “generally rectangular cross-section”, as used herein, refers to a ring structure which extends partially or fully around the surgical site, which is characterized by a generally planar major surface generally in the plane of the ring, together with opposing corners defining the opposite major surface of the ring, along a substantial majority of its length. The outer side of the ring of the Bookwalter/Codman typically has regularly-spaced arc-shaped notches, but the notches do not prevent the Bookwalter/Codman ring from having a generally rectangular cross-section. Other cross-sectional shapes, including trapezoidal cross-sectional shapes, cross-sectional shapes having an inside thickness which differs from the outside thickness of the ring (see, for instance, FIG. 9 of U.S. Pat. No. 4,434,791), shapes having a concave major surface which still defines a plane, or shapes having a slightly convex major surface which still provides the opposing corners, are also of generally rectangular cross-section.

In the Bookwalter/Codman system and in some of these other examples, a support clamp is used to secure the generally rectangular stock of the ring to the support post. While two or more support clamps can be used from opposing directions to support the ring between different support points, in many retractor arrangements a singular support clamp is used to cantilever the ring over the surgical site. For the retractor system to work most effectively, the support clamp must allow quick tightening and loosening for fast assembly and disassembly of the system. The support clamp when loosened should allow a wide range of movement and placement of the ring, but when tightened the support clamp should securely hold the ring in place. The retractor blades are connected to the inner side of the ring, and support clamps can also be used to attach retractor blades to the generally rectangular retractor ring.

### BRIEF SUMMARY OF THE INVENTION

The present invention is a clamp for attachment of bar stock of generally rectangular cross-section, particular for use in surgical retractor systems, such as for attaching to a Bookwalter/Codman ring. The clamp has an inventive attachment mechanism that interacts with corners of the generally rectangular cross-section and permits clamping against a wide range of widths and thicknesses while still retaining a simple and intuitive tightening action. The clamp separately absorbs the clamping stress as a hoop stress, actuated with a mechanical advantage on the tightening control. In the preferred arrangement, the clamp makes a tripod top/planar bottom contact with the generally rectangular bar stock of the ring. An inventive articulating joint permits best fold up of the clamp, with a ball shaft which is constrained within the bisecting plane of the joint clamp.

### BRIEF DESCRIPTION OF THE DRAWINGS

FIG. 1 is a perspective view of one preferred embodiment of a support clamp in accordance with the present invention, shown attached to exemplary rectangular bar stock.

FIG. 2 is an exploded perspective view of the support clamp of FIG. 1.

FIG. 3 is a side view of the pivot clamp sleeve of the support clamps of FIGS. 1-2.

FIG. 4 is an end view of the pivot clamp sleeve of FIG. 3.

FIG. 5 is a cross-sectional view of the pivot clamp sleeve taken along line 5-5 of FIGS. 3 and 4.

FIG. 6 is an enlarged cross-sectional side view of the head assembly of FIGS. 1-2, showing the range of rectangular bar stock which may be encountered for the ring structure.

FIG. 7 shows a cross-sectional view of the head assembly of FIG. 6 tightening on the exemplary rectangular bar stock.

FIG. 8 shows a cross-sectional view of the head assembly of FIGS. 6 and 7 in a cleaning position.

FIG. 9 is an exploded perspective view of a second preferred embodiment of a support clamp in accordance with the present invention.

FIG. 10 is a perspective view of another preferred embodiment of a support clamp in accordance with the present invention, shown attached to exemplary rectangular bar stock.

FIG. 11 is an exploded perspective view of the alternative head assembly of FIG. 10.

FIG. 12 is a cross-sectional side view of the head assembly of FIGS. 10 and 11 during tightening.

While the above-identified drawing figures set forth preferred embodiments, other embodiments of the present invention are also contemplated, some of which are noted in the discussion. In all cases, this disclosure presents the illustrated embodiments of the present invention by way of representation and not limitation. Numerous other minor modifications and embodiments can be devised by those skilled in the art which fall within the scope and spirit of the principles of this invention.

### DETAILED DESCRIPTION

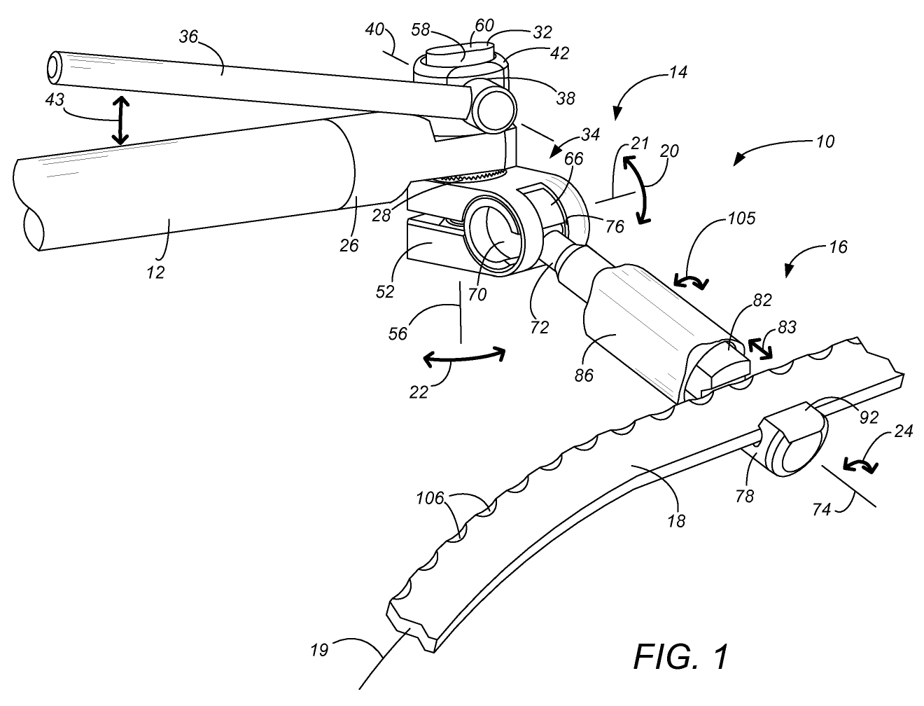

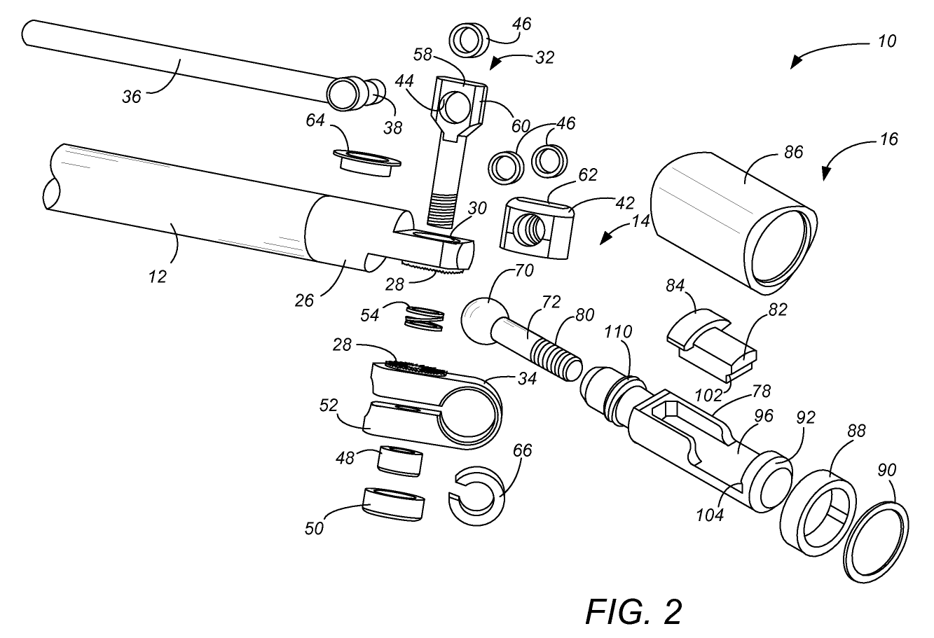

A first embodiment of a support clamp assembly 10 of the present invention is shown in FIGS. 1 and 2. The support clamp assembly 10 includes three basic components: an arm 12, an articulating joint 14, and a support clamp 16. The support clamp 16 attaches to the generally rectangular cross-section of the ring 18, a portion of which is shown in FIG. 1. The ring 18 may be any of the rings disclosed in U.S. Pat. Nos. 1,919,120, 1,963,173, 4,254,763, 4,421,108, 4,424,724, 4,434,791, 4,467,791, 5,375,481, 5,520,608, 5,520,610, 6,241,659, 6,530,882 and 6,808,493, all incorporated by reference. Each of these rings are generally provided by a bar having a longitudinal axis 19 and a generally rectangular cross-section transverse to the longitudinal axis.

As known in the art, the function of the arm 12 is to extend, most commonly horizontally, from a support post (not shown, typically mounted vertically on a bed frame) toward a surgical site location desired for the ring 18. The function of the articulating joint 14 is to permit adjustment of the ring pitch angle 20 about axis 21, yaw angle 22 about axis 56 and/or roll angle 24 about axis 74, each relative to the arm 12.

The arm 12 can be any strong structure as known in the art, and the construction of the arm 12 and its attachment to the support post or the bed frame is not of particular significance here. The preferred arm 12 is a seamless tubular structure of a sterilizable material such as a 17-4 stainless steel. A preferred size is about 10-12 inches long, at an outer diameter of about 1 inch and a wall thickness of about ⅛ of an inch.

A serrated tube tip 26 can be welded to or otherwise integrally joined or formed on a distal end of the arm 12. The preferred tube tip 26 extends for a length of about ½ inch inside the arm 12 (extension not shown), enabling a strong, rigid, welded connection. The preferred tube tip 26 adds a length of about 2 inches to the length of the arm 12. The serrations 28 may extend radially about an opening 30 for a clamp bolt 32. For instance, preferred serrations 28 have an outer diameter of about 0.9 inches, an inner diameter of about 0.65 inches, and a serration height of around 1/16 of an inch.

The serrations 28 on the tube tip 26 mate with mirror image serrations 28 on the top of a serrated joint clamp 34. The serrated or toothed attachment between the serrated joint clamp 34 and the serrated tube tip 26 allows 360° rotational placement of the serrated joint clamp 34 (yaw 22 of the retractor ring 18) relative to the arm 12. While some degree of yaw 22 is important for alignment flexibility of the retractor ring 18, a full 360° rotational placement is beneficial for “fold up” storage of the support clamp assembly 10 in a small location such as a surgical tray (not shown). The preferred serrations 28 permit stepped rotational placement of the joint clamp 34 relative to the arm 12, in stepped increments selected from about 1 to 10°. The preferred stepped increment is about 5°. The serrations 28 provide a secure attachment so the joint 14 can support a considerable moment on the ring 18 relative to the arm 12 without slipping. Alternatively, the tube tip 26 and the joint clamp 34 may mate at a frictional surface which permits continuous rather than stepped adjustment, although such frictional attachment tends not to be able to support as much moment without slippage.

The joint clamp 34 is tightened with a clamp tightening handle 36 which is preferably accessible on the top of the support clamp assembly 10. The first preferred handle 36 is merely a bar structure which rotates a cam 38 about a generally horizontal axis 40 defined in a cam body cap 42. The preferred throw 43 of the handle 36 is downward so the handle 36 is tightened into a parallel arrangement just above the arm 12. The cam 38 mates with a circular opening 44 in a clamp bolt 32, such that rotation of the cam 38 raises the clamp bolt 32 to tighten the joint clamp 34. Bearing rings 46 may provide low friction bearing surfaces between the cam 38 of the handle 36 and the cam body cap 42 and clamp bolt 32.

A nut 48 and a nut cap 50 are preferably used to secure the threaded end of the clamp bolt 32 relative to the bottom leg 52 of the joint clamp 34. The clamp bolt 32 has sufficient length and diameter to transfer the clamping force from the handle 36 to the joint clamp 34. In the preferred embodiment, the clamp bolt 32 has a diameter of about ⅜ inch and a length of about 2 inches.

A compression spring 54 may be disposed in the clamp about the clamp bolt 32. The compression spring 54 provides a biasing force between the joint clamp 34 and the tube tip 26, so when the handle 36 is loosened the serrated connection opens up to allow rotation of the joint clamp 34 relative to the arm 12.

The clamp tightening handle 36 may be retained aligned with the arm 12, but alternatively may rotate about the (vertically shown) axis 56 of the clamp bolt 32. If alignment between the arm 12 and the clamp tightening handle 36 is desired, the cam body cap 42 is rigidly fixed to the tube tip 26. Flats 58 on the head 60 of the clamp bolt 32 mate with corresponding flats 62 on the cam body cap 42, so the clamp tightening handle 36 always tightens to a position parallel to and above the arm 12. If rotation of the clamp tightening handle 36 about the clamp bolt axis 56 is desired, the cam body cap 42 can be rotationally attached to the tube tip 26. The rotational attachment of the cam body cap 42 to the tube tip 26 allows the surgeon to orient the handle/handle throw plane at an angle to the arm 12, beneficial for instance if clearance over the arm 12 is limited in the surgical arena. A horizontally oriented bearing ring 64 may be used to reduce rotational friction between the cam body cap 42 and the tube tip 26.

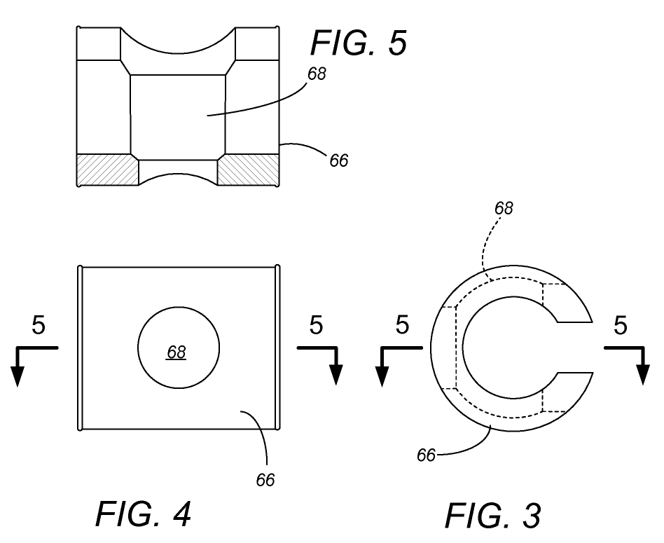

The articulating joint 14 of the present invention is preferably a ball-in-socket joint using a ball/ball shaft in a sleeve 66, with the sleeve 66 further shown in FIGS. 3-5. The sleeve 66 is shaped with a recess 68 matching the diameter of the ball 70, to receive the ball 70 therein. The sleeve 66 is preferably formed of a wear-resistant alloy such as NITRONIC 60, while the remaining components of the articulating joint 14 can be formed of an appropriately strong sterilizable material such as surgical stainless steel. The sleeve 66 is preferably formed with an overall “C” shape in cross-section, so tightening of the clamp compresses the sleeve 66 about the outer diameter of the ball 70. The ball 70 has a sufficient outer diameter so tightening of the joint clamp 34 will secure the ball/ball shaft relative to the joint clamp 34. In the preferred embodiment, the ball 70 has a spherical outer diameter of about ⅔ of an inch.

The ball-in-socket joint 14 permits pitch 20 and roll 24 adjustments to the ball shaft 72, but the ball shaft 72 always stays aligned with the central bisecting plane of the clamp 34, that is, the axis 74 of the support clamp 16 always intersects the clamp bolt axis 56. Alignment between the ball shaft 72 and the central bisecting plane of the clamp 34 is achieved by having the ball shaft 72 extend through a central opening 76 in the clamp 34. The ball-in-socket joint 14 does not permit yaw adjustment of the shaft 72, which is solely provided by the serrated connection on the joint clamp 34. In the preferred embodiment, the central opening 76 in the clamp 34 is a slot 0.38 inches in width. The ball shaft 72 has a diameter of 0.376 inches, mating with the 0.38 inch central opening 76 to maintain alignment with a minimal overall width of about 1 inch. As such, the clamp 34 when loosened can easily fold up into a generally flat arrangement of only 1¼ inch in thickness, enhancing the “fold up” feature of the support clamp assembly 10.

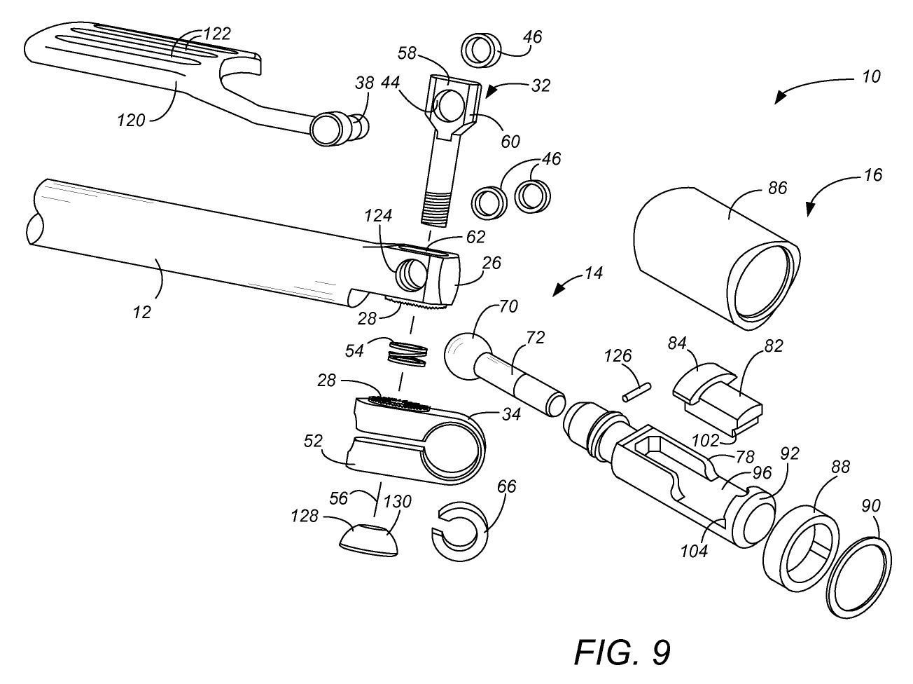

During clamping, the serrated clamp 34 tightens the sleeve 66 onto the ball 70 of the support clamp 16. The support clamp 16 or head assembly includes a clamp head 78 fixedly attached to the ball shaft 72. For instance, the fixed attachment of the ball shaft 72 to the clamp head 78 may be by welding, with a tight press fit, or with an attachment pin 124 (shown in FIG. 9) or set screw. The preferred embodiment shown in FIG. 2 merely uses a threaded connection 80 between the ball shaft 72 and the clamp head 78. Operation of the support clamp 16 does not involve any movement of the clamp head 78 relative to the ball shaft 72, so the clamp head/ball shaft combination could alternatively be formed as a single component. Forming the ball shaft 72 separately from the clamp head 78 assists in ease of machining, which is the simplest (but not exclusive) method of forming both the ball shaft 72 and the clamp head 78, as well as in assembly with the joint clamp 34. The overall length of the ball shaft 72 is selected to provide sufficient clearance of the support clamp 16 from the joint clamp 34 through the opening 76, and to provide sufficient length for rigid attachment of the clamp head 78 to the ball shaft 72. In the preferred embodiment, the threads 80 extend over a length of about ⅔ of an inch on the ball shaft 72, and another about ⅔ inch length of the ball shaft 72 is provided for clearance through the joint clamp opening 76.

A clamping jaw 82 moves relative to the clamp head 78 as shown by arrows 83 to provide the clamping force on the generally rectangular cross-section ring 18. The clamping jaw 82 should have sufficient width to provide some error in placement and shape of the ring 18. For instance, the preferred clamping jaw 82 has a width of about ½ inch. A push shoulder 84 is provided on the proximal end of the clamping jaw 82 for biasing the clamping jaw 82 forward toward the distal end of the clamp head 78. Preferred dimensions of the push shoulder 84 are about ⅜ inch in length and about 7/12 inch in height, i.e., about 1⅙ inch in outer diameter relative to the axis 74 of the clamp head 78

A locking knob collar 86 provides a mechanical advantage and hand tightenable control for movement of the clamping jaw 82. The preferred locking knob collar 86 extends for a length of about 2 inches, with an outer diameter of about 1¼ inches. This diameter is as small as possible so the clamp assembly 10 is as unobtrusive as possible in the surgical arena, while still providing a sufficient diameter and grasping surface area for hand torqueing to tighten the clamp 16. The locking knob collar 86 preferably has an exterior structure to facilitate grasping and tightening of the locking knob collar 86. The locking knob collar 86 need not have a cylindrical outer profile, but rather may include some knurls, texture or shape to facilitate hand torqueing of the locking knob collar 86. In the preferred configuration shown in FIGS. 1 and 2, a generously radiused triangular cross-sectional shape facilitates twisting of the locking knob collar 86 about the axis 74 of the clamp head 78.

A spacer 88 and retaining ring 90 are used for anti-friction biasing of the clamping jaw 82 and to complete assembly of the support clamp 16. As shown, the spacer 88 can be formed of an anti-friction or lubricious material such as PEEK, while the other components of the head assembly can be formed of an appropriately strong sterilizable material such as surgical stainless steel.

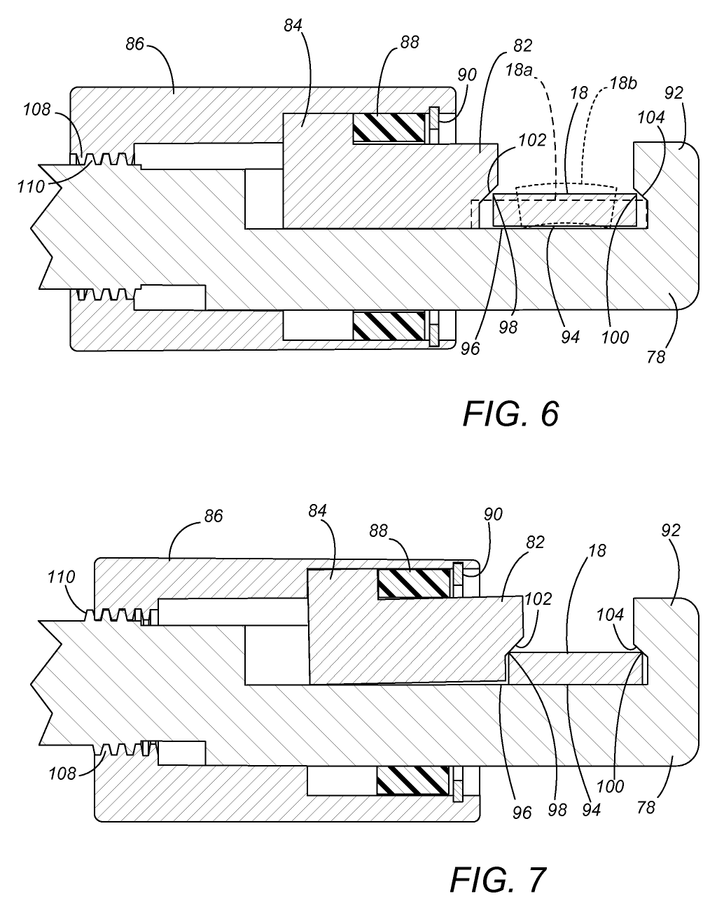

Operation of the support clamp 16 is best shown in the cross-sectional views of FIGS. 6-8. The locking knob collar 86 is rotated to retract the clamping jaw 82 away from the clamping distal end 92 of the clamp head 78. The ring 18 of the retractor system is placed into the support clamp 16.

As shown in FIG. 6, the ring 18 of the support clamp 16 may have any of a range of dimensions and still be of generally rectangular cross-section. The examples shown in dashed lines include a relatively thin, wide ring 18a and a relatively thick, concave/convex trapezoidal ring 18b. All of these cross-sections have a bottom surface 94 (a major leg of the cross-sectional shape) which generally defines a plane, and thus makes aligning contact with a planar base contact area 96 of the clamp head 78. All of these cross-sections also have two spaced top corners 98, 100 which characteristically define the cross-sectional shape. As long as the two spaced top corners 98, 100 of the cross-sectional shape are each at a height from the bottom surface 94 such that each falls within the slope of its corresponding corner contact area 102, 104 of the clamp head 78 and clamping jaw 82, the support clamp 16 can handle a wide range of cross-sectional sizes.

This ability for the clamp head 78 to handle a wide range of cross-sectional shapes and sizes is important particularly in retrofitting against retractor rings 18 which may already be in use in the marketplace. For instance, the Bookwalter/Codman systems existing in numerous hospitals and surgery rooms throughout the country include rings 18 which may be designed differently in term of the thickness and width of the ring 18. Bookwalter/Codman rings 18 which are nominally dimensioned identically may be of different thickness and/or widths if tolerances were not tightly kept during manufacture of such rings 18. Even a single ring 18 may have different thicknesses or different widths along the length of the ring 18. Many Bookwalter/Codman rings include notches 106 in the ring 18 as shown in FIG. 1, but are still of generally rectangular cross-sectional shape. The support clamp 16 of the present invention is sufficiently flexible to permit attachment quickly and tightly to all such rings 18 within a wide dimensional range, even without knowing the exact thickness, width and/or cross-sectional shape of the ring 18 prior to manufacture of the support clamp 16.

As best shown in FIGS. 6-8, the preferred clamps have corner contact areas 102, 104, one each on the clamping jaw 82 and on the clamp head 78, which slope at 45°. These sloped contact areas 102, 104 extend at heights of from about 0.125 to 0.225 inches above the planar base contact area 96. The preferred clamps 16 can according receive Bookwalter/Codman rings 18 that have a thickness of their inside edge of anywhere between about 0.125 and 0.225 inches, and a thickness at their outer edge of any where between about 0.125 and 0.225 inches.

The locking knob collar 86 is rotated relative to the clamp head 78 (as shown by arrows 105 in FIG. 1) to push the clamping jaw 82 forward toward the ring 18. As the clamp 16 tightens on the ring 18, the distally positioned corner 100 of the ring cross-section (i.e., the upper right corner in the orientation shown in FIGS. 6-7) contacts somewhere along the height of a fixed corner contact 104 on the clamp head 78. The fixed corner contact 104 on the clamp head 78 provides a stop which presses against the ring 18 when the clamp 16 is tightened. The opposing proximally positioned corner 98 of the ring cross-section (i.e., the upper left corner in the orientation shown in FIGS. 6-7) contacts somewhere along the height of the moving corner contact 102 on the clamping jaw 82. The fixed corner contact 104 and the moving corner contact 102 are both angled relative to the base surface 96, so tightening of the clamp 16 biases the ring 18 downward into the base plane 96 of the clamp head 78. As the clamping jaw 82 moves further forward during tightening, the angled orientation of the moving corner contact 102 causes the clamping jaw 82 to rotate slightly (counter-clockwise in FIG. 7) under the moment placed upon it by the ring 18. With the preferred embodiment dimensions, the clamping jaw 82 can rotate in a range up to about 2 or 2.5° before fully binding up the clamp 16. With the slight rotation of the clamping jaw 82, the clamping jaw 82 compresses into the top side of the spacer 88 and into the top side of the locking knob collar 86.

The spacer 88 and the locking knob collar 86 each absorb the compressive force of the clamping jaw 82 in a hoop stress wrapping around the clamp head 78. While the clamp is tightened, the vast majority of the clamping force is bourn by this hoop stress rather than by the threaded connection between the locking knob collar 86 and the clamp head 78. The ratio between clamping hoop stress and stress on the threaded connection can be selected as desired by choosing the angle of the moving corner contact 102, with a preferred angle being 45°. This angle, together with the advance length (a preferred value of 0.100 inches), are selected by balancing the desired tightening torque to clamp force ratio and the additional length for the support clamp 16 required to accept the thickness variance of the retractor rings 18 with which the clamp 16 may be used.

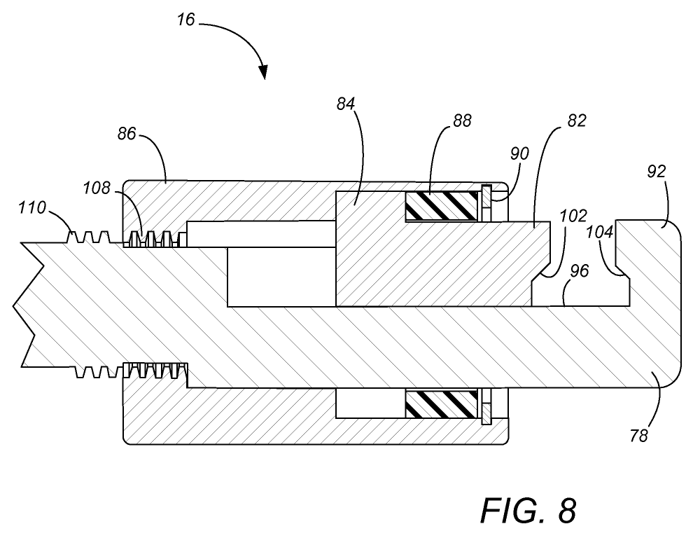

A separate feature of the preferred support clamp 16 is depicted in FIG. 8, which shows a cleaning position of the support clamp 16. With no ring 18 in place, the locking knob collar 86 can be rotationally advanced until its threads 108 fully disengage from the threads 110 of the clamp head 78. With the threads 108, 110 fully disengaged, cleaning and sterilization of the preferred support clamp 16 is easier. Additionally, when the support clamp 16 is loosened, clearance tolerances exist between the clamping jaw 82 and the spacer 88, between the clamping jaw 82 and the locking knob collar 86, and between the spacer 88 and the locking knob collar 86. These clearances not only facilitate cleaning and sterilization, but further ensure unrestricted travel of the clamping jaw 82 until it binds up on the retractor ring 18. In the preferred embodiment, the clamping jaw/clamp head have a combined small section diameter of 0.875 inches or less, mating against the inner diameter of the spacer 88 of 0.885 inches, for a clearance of about 0.01 inches. In the preferred embodiment, the clamping jaw 82 has a large section diameter of 1.18 inches, mating against the inner diameter of the locking knob collar 86 of 1.19 inches, for a clearance of about 0.01 inches. In the preferred embodiment, the locking knob collar 86 has an inner diameter of 1.19 inches while the spacer 88 has an outer diameter of 1.18 inches for a clearance of about 0.01 inches.

As an alternative to having the cleaning position shown in FIG. 8, either the threads 108 on the locking knob collar 86 can be made to extend further proximally or more preferably the threads 110 on the clamp head 78 can be made to extend further distally. By having the threads 108 or 110 extend further, the locking knob collar 86 can be advanced to push the clamping jaw 82 further distally, all the way until the moving corner contact 102 abuts the fixed corner contact 104.

The preferred locking knob collar 86 provides its mechanical advantage through a threaded connection 108, 110 with the clamp head/ball shaft. As examples, this may be a single helical thread as depicted in the embodiment of FIGS. 6-8, or a double helical thread (not shown). By rotation of the locking knob collar 86 relative to the clamp head 78, the locking knob collar 86 pushes the clamping jaw 82 forward (transverse to the longitudinal axis 19 of the ring 18) and into engagement with a cross-sectional corner 98 of the retractor ring 18. Many other mechanisms for advancing the clamping jaw 82 would also function, but the preferred embodiment provides the advancing linkage with the same structure (the locking knob collar 86) which absorbs the hoop stress of binding, in a way that is elegant, easy to manufacture, inherently understandable and easy to use.

An alternative embodiment of the support clamp assembly of the present invention is shown in FIG. 9. This alternative embodiment includes four primary differences relative to the embodiment of FIGS. 1-8, related to the handle 36, to the cam body cap 42, to the attachment of the clamp head 78 to the ball shaft 72, and to the clamp bolt nut 48. Other alternative embodiments include various combinations of one, two or three of these four features.

In the embodiment of FIG. 9, the handle 120 is not a simple cylindrical bar shape, but rather is a wider, flat “waffle” shape, with openings 122 through the broad surface of the handle 120. The wider size of the handle 120 allows the surgeon to more comfortably press the handle 120 downward with significant torque into a tight, locked position for the clamp 34. The wider size of the handle 120 also centers the handle directly over the arm 12, for an intuitive, scissors-type clamping of the handle 120 down toward the arm 12.

The openings 122 in the handle 120 reduce the weight and amount of metal or other material used to form the handle 120. More importantly, the openings 122 in the handle 120 help to conduct heat from the handle 120 to surrounding air. The heat conduction rate from the handle 120 is important particularly in situations where the support clamp assembly is heat sterilized, such as in a heated autoclave, immediately prior to use. Quick conduction of heat from the handle 120 is important so the surgeon does not burn his or her hand or gloves while tightening the handle 120, without requiring a waiting time after autoclaving for heat to escape from the support clamp assembly 10.

A second difference in the clamp assembly of FIG. 9 is that there is no separately formed cam body cap 42, but rather openings 124 for the cam 38 are disposed directly into the tube tip 26. This results in fewer total parts and easier assembly of the support clamp assembly 10.

A third difference in the clamp assembly of FIG. 9 is that the ball shaft 72 is not threadably connected to the clamp head 78, but rather is press fit and secured with a press pin 126 through a hole (not shown) in the clamp head 78.

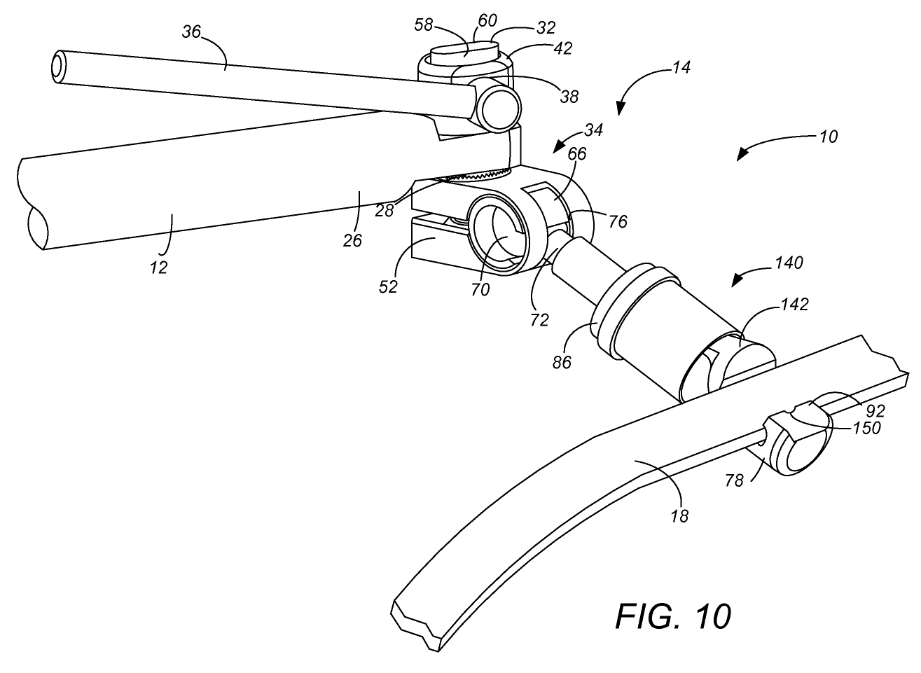

A fourth difference in the clamp assembly of FIG. 10 is a different construction of the clamp bolt nut. In particular, the clamp bolt nut 128 of this embodiment is formed with a spherical contact surface 130, which mates with a spherical recess in the lower leg 52 of the joint clamp 34. The spherical mating relationship keeps the joint clamp 34 from binding as the orientation of the clamp bolt 32 changes slightly during the rotational movement of the cam 38 during tightening. The spherical clamp bolt nut 128 thus provides for a smoother tightening action on the joint clamp 34. As one alternative to the spherical nut 128, the clamp bolt head 60 could have a spherical bottom surface which mates with the top of the tube tip 26 and permits some orientational adjustment of the clamp bolt axis 56 relative to the tube tip 26 and joint clamp 34. As another alternative to a spherical nut 128, the clamp bolt 32 could be positioned “upside down”, and have a spherical head that mates with the bottom leg 52 of the joint clamp 34. In any of these arrangements, the important aspect is a spherical surface transmitting the force of the clamp bolt 32, which allows some change of orientation of the clamp bolt 32 during tightening. The permitted change of orientation of the clamp bolt 32 is particularly important in this embodiment of FIG. 9 which has no separately formed cam body cap 42, i.e., when there is no play or permitted movement of the cam body cap 42 relative to the tube tip 26 during tightening.

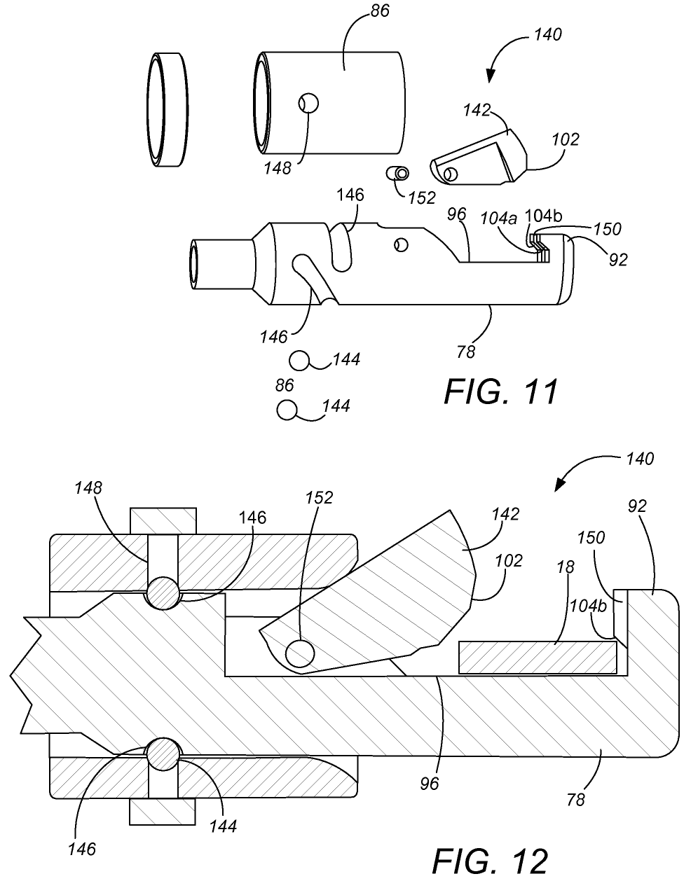

A second alternative embodiment of the support clamp assembly is shown in FIGS. 10-12. This embodiment 140 of the support clamp is similar to the support clamps 16 of FIGS. 1-9, but differs in three significant respects. First, the support clamp 140 of FIGS. 10-12 uses a different advancement mechanism for the locking knob collar 86. Second, the second embodiment uses a hinged clamping jaw 142 rather than a linearly advancing clamping jaw 82. Third, the shape of the clamp head 78 facilitates a three point/planar contact with the ring 18. Alternative embodiments include various combinations of one or two of these three features.

Rather than use a threaded connection between the locking knob collar 86 and the clamp head 78, the advancement mechanism of the embodiment of FIGS. 10-12 includes one or more guide projections 144 which mate into an equal number of rotationally oriented slots 146. In the preferred embodiment, the guide projections are ball guides 144 positioned in recesses 148 on the locking knob collar 86, and the slots 146 are disposed on the proximal side of the clamp head 78. Alternatively, guide projections could be disposed on the clamp head 78 which mate into slots on the locking knob collar 86.

The slots 146 for the guide projections 144 do not have a constant pitch angle, but rather allow advancement of the locking knob collar 86 at a varying advance rate (and thus a varying mechanical advantage). In the preferred embodiment, two rotationally oriented slots 146 are provided, spaced 180° from each other, which each extend 180° helically around the clamp head 78. The first 60° of rotation results in a 0.283 inch advancement of the locking knob collar 86. Another 60° of rotation results in an additional 0.142 inch advancement of the locking knob collar 86, such that the first 120° of rotation results in a 0.425 inch advancement. A further 60° of rotation results in an additional 0.049 inch advancement of the locking knob collar 86, such that 180° of rotation results in a total advancement of 0.474 inch. That is, the non-constant advance rate has a greater amount of relative advancement when the clamping contact is at a far, loosened position relative to the stop 92 and a lesser amount of relative advancement when the clamping contact is at a near, tightened position relative to the stop 92. The non-constant advance rate smoothly changes from the greater amount of relative advancement to the lesser amount of relative advancement as the grasping collar 86 is rotationally advanced. By using a differing advance rate, the support clamp 140 binds up on most rings 18 with a shorter rotation of the locking knob collar 86 of less than 180° and/or a greater mechanical advantage (optimally designed at 150° of rotation to bind on the most common, nominal size of a Bookwalter/Codman ring 18). Other linkage mechanism can alternatively be used to provide a non-constant mechanical advantage for biasing the moving contact against the ring 18, such as vice grip pliers or cammed types of linkages well known in the clamping arts.

In the embodiments of FIGS. 10-12, the advancement of the locking knob collar 86 results in a pivoting of the pivoting clamping jaw 142 rather than a sliding of the clamping jaw 82. The pivot pin 152 of the pivoting clamping jaw 142 does not change its position relative to the clamp head 78. Accordingly, as the locking knob collar 86 advances but the pivot axis of the pivoting clamping jaw 142 remains stationary, the locking knob collar 86 act as a pusher for the pivoting clamping jaw 142 which is in contact with a differing location of the pivoting clamping jaw 142. That is, the contact point moves relative to the pivoting clamping jaw 142 as the locking knob collar 86 slides up the pivoting clamping jaw 142, and the contact point moves relative to the locking knob collar 86 as the pivoting clamping jaw 142 changes its angle relative to the locking knob collar 86. Because this contact location varies continuous during advancement, the distal end of the locking knob collar 86 should have a generously radiused inside corner on its edge, and the top surface of the pivoting clamping jaw 142 should also be generously radiused.

Because the pivoting clamping jaw 142 pivots rather than translates, its contact point with the generally rectangular stock of the ring 18 also changes location. The preferred pivoting clamping jaw 142 has a contact plane which ranges from an angle of about 55 to 45° relative to the base plane as it contacts the proximal cross-sectional corner 98 of the ring 18 and clamps onto the generally rectangular cross-sectional shape of the ring 18.

While the embodiments shown include several preferred linkages for moving the clamping jaw 82, 142 against the ring 18, workers skilled in the art will appreciate that many other types of linkages could alternatively be used. One particular benefit of the linkages shown is that the rotational force placed upon the locking knob collar 86 is well balanced relative to the clamp head 78 and relative to the ring 18. With a well balanced tightening force, it is much easier for the surgeon to tighten down the support clamp assembly in the desired position of the ring 18.

The third significant difference between the embodiment of FIGS. 1-8 and the embodiments of FIGS. 10-12 is brought out by an arc shaped recess 150 in the clamp head 78. With this arc shaped recess 150, the locations where the clamp head 78 contacts the generally rectangular stock of the ring 18 are purposefully separated, such that all rings 18 will arrange themselves with a tripod top/planar bottom contact into the clamp 16, i.e., two contact locations 104a, 104b on the clamp head 78 on opposite sides of the arc shaped recess 150, and a third contact location 102 on the moveable clamp jaw 82. By separating the contact points on the top corners 98, 100 of the ring 18 into a triangle, with each vertex of the triangle pushing down toward the planar bottom 96, the clamp 16 makes a tight connection with a wide variety of Bookwalter/Codman rings 18, similar to the way a three-legged stool will sit on a planar floor without rocking. Further, the tight connection is made without regard to whether the clamp 16 is attached to a straight side or a curved side of the Bookwalter/Codman ring 18, and without regard to strict tolerances or the design of the Bookwalter/Codman ring 18.

Although the present invention has been described with reference to preferred embodiments, workers skilled in the art will recognize that changes may be made in form and detail without departing from the spirit and scope of the invention.

1. A clamp for attaching to a bar of a surgical retractor support system, the bar having a generally rectangular cross-section, the clamp comprising:
a fixed corner contact for contacting a first corner of the generally rectangular cross-section;
a base contact for contacting a leg of the generally rectangular cross-section opposite the first corner, the first corner contact and the base contact being fixed with respect to each other;
a moving corner contact for contacting a second corner of the generally rectangular cross-section, the moving corner contact being movable relative to the fixed corner contact and the base contact; and
a linkage for biasing the moving corner contact against the second corner, thereby clamping the bar between the fixed corner contact, the base and the moving corner contact.

2. The clamp of claim 1, wherein the moving corner contact pivots relative to the base contact and fixed corner contact.

3. The clamp of claim 2, wherein the linkage provides a non-constant mechanical advantage in biasing the moving corner contact.

4. The clamp of claim 1, wherein the moving corner contact translates relative to the base contact and fixed corner contact.

5. The clamp of claim 1, wherein the base contact defines a generally a planar contact area for a generally planar surface of the bar, and wherein the fixed corner contact and the moving corner contact comprise a triangle of generally point contact locations against two edges of the bar opposite the generally planar surface of the bar.

6. A method for attaching a bar to a surgical retractor support system, the method comprising:
inserting the bar into an opening in a clamp of the surgical retractor support system, the opening being defined between a fixed stop and a pivoting contact member of the clamp; and
pivoting the pivoting contact member into biased engagement with the bar, thereby clamping the bar between the pivoting contact member and the fixed stop.

7. A clamp for attaching to a bar of a surgical retractor support system, the bar extending along a longitudinal axis, the clamp comprising:
a stop for contacting a first side of the bar on one side of the longitudinal axis;
a bearing rod fixed relative to the stop, the bearing rod defining a tightening axis;
a grasping collar rotatably mounted on the bearing rod for rotation about the tightening axis; and
a clamping contact for contacting a second side of the bar on an opposing side of the longitudinal axis to thereby clamp the bar between the clamping contact and the stop, with rotational movement of the grasping collar relative to the bearing rod advancing the clamping contact toward the stop.

8. The clamp of claim 7, wherein, the grasping collar is coupled to the bearing rod such that rotation of the grasping collar relative to the bearing rod advances the grasping collar along the tightening axis and relative to bearing rod; and wherein advancement of the grasping collar advances the clamping contact toward the stop.

9. The clamp of claim 7, wherein rotation of the grasping collar relative to the bearing rod advances the clamping contact toward the stop at a varying advance rate, and further comprising:
an advancement slot wrapped about the longitudinal axis and provided on one of the bearing rod and the grasping collar, the advancement slot having a curvature which provides a non-constant advancement slope; and
a projection provided on the other of the bearing rod and the grasping collar, the projection riding in the advancement slot.

10. A method of tightening a clamp for attaching to a clamp to a bar of a surgical retractor support system, the method comprising:
rotating a grasping collar about a bearing rod about a tightening axis of rotation, the rotation causing the grasping collar to advance along the tightening axis of rotation at a non-constant advance rate relative to bearing rod, the advance of the grasping collar being coupled relative to the clamp to tighten the clamp onto the bar.

11. A clamp for attaching to a bar of a surgical retractor support system, the bar extending along a longitudinal axis, the clamp comprising:
a stop for contacting a first side of the bar on one side of the longitudinal axis;
a clamping contact member for contacting a second side of the bar on an opposing side of the longitudinal axis to thereby clamp the bar between the clamping contact member and the stop; and
a collar for biasing the clamping contact member toward the bar, the collar fully encircling at least a portion of the stop and at least a portion of the clamping contact member, such that a static clamping force for the clamp is absorbed as a hoop stress on the collar.

12. The clamp of claim 11, wherein the collar is advanced along an advancement axis to bias the clamping contact member toward the bar, wherein the advancement axis is generally transverse to the longitudinal axis of the bar.

13. The clamp of claim 11, wherein the collar is rotationally advanced along an advancement axis to bias the clamping contact member toward the bar.

14. The clamp of claim 11, wherein the contact point between the collar and the clamping contact member changes relative to at least one of the collar and the clamping contact member during advancement of the clamping contact member toward the bar.

15. The clamp of claim 14, wherein the contact location is provided by a curved leading inside edge on the collar.

16. A joint for a surgical retractor support system, comprising:
a support arm having a distal end;
a clamp attached at the distal end of the support arm;
a ball-in-socket joint within the clamp, with a shaft extending from the ball-in-socket joint, the ball-in-socket joint permitting roll and pitch adjustment of the shaft but not permitting yaw adjustment of the shaft.

17. The joint of claim 16, further comprising a serrated joint on the clamp which permits 360° yaw adjustment of the ball-in-socket joint relative to the support arm.

18. The joint of claim 16, wherein the shaft is constrained within a bisecting plane of the clamp.

19. A joint for a surgical retractor support system, comprising:
first and second legs on opposing sides of a clamp recess;
a clamp bolt which biases one of the first and second legs to change the size of the clamp recess, the clamp bolt having a spherical contact surface coupled to one of the first and second legs to provide a clamping force.

20. The joint of claim 19, further comprising a cam for biasing the clamp bolt relative to the other of the first and second legs.
